(19) United States
(12) Patent Application Publication
(10) Pub. No.: US 2012/0264693 A1
(43) Pub. Date: Oct. 18, 2012

(54) COMPOUNDS ACTING AS PEPTIDE GAP JUNCTION MODULATORS, AND USES THEREOF

(75) Inventors: Mario Delmar, Ann Arbor, MI (US); Steven M. Taffet, Syracuse, NY (US); Wanda Coombs, New York, NY (US); Vandana Verma, Ann Arbor, MI (US); Bjarne Due Larsen, Roskilde (DK)

(73) Assignees: Zealand Pharma A/S, Glostrup (DK); The Regents of the University of Michigan, Ann Arbor, MI (US); The Research Foundation of State Univesity of New York, Albany, NY (US)

(21) Appl. No.: 13/377,773

(22) PCT Filed: Jun. 11, 2010
(86) PCT No.: PCT/DK2010/000092

§ 371 Date: Jun. 28, 2012

Related U.S. Application Data
(60) Provisional application No. 61/187,839, filed on Jun. 17, 2009.

(30) Foreign Application Priority Data
Jun. 12, 2009 (EP) 09007772.8

(51) Int. Cl. A61K 38/12; C12N 5/07; A61P 9/00; A61P 17/00; A61P 27/02; A61P 13/10; A61P 19/10; A61P 13/12; A61P 35/00; A61P 19/00; A61P 13/02; C07K 7/64; A61P 25/00

(52) U.S. Cl. 514/15.4; 530/317; 514/21.1; 435/375; 514/16.4; 514/18.6; 514/20.8; 514/17.7; 514/16.9; 514/19.3

(57) ABSTRACT

Compounds capable of modulating intracellular gap junctional communication, as well as their use in the treatment of diseases associated with impaired gap junction intracellular communication (GJIC) 1 are disclosed.

### FIELD OF THE INVENTION

The invention relates to compounds capable of modulating intracellular gap junctional communication, to pharmaceutical compositions comprising the compounds and their medical uses for the prevention and/or treatment of conditions associated with modulating gap junction communication.

### BACKGROUND OF THE INVENTION

There is increasing recognition that intercellular communication is essential for cellular homeostasis, proliferation and differentiation. Such communication is believed to be facilitated by gap junctions. These structures are thought to be a route for coupling cells and permitting “cross-talk”. See generally, Sperelakis, N., (1989) Cell Interactions and Gap Junctions by N. Sperelakis, William C. Cole (Editor). Connexins are integral membrane proteins that oligomerize to form intercellular channels called gap junctions. The most abundant gap junction protein in a number of mammalian systems is connexin43 (“Cx43”).

Gap junction channels are responsible for direct cell-to-cell communication. These channels are dynamic pores that are regulated in response to changes in the cellular environment and by protein interactions. In the heart, gap junction channels are critical structures in the mechanism for passage of electrical impulses (Lerner et al., “Accelerated Onset and Increased Incidence of ventricular arrhythmias induced by ischemia in Cx43-deficient mice,” Circ. 101(5):547-552 (2000); Gutstein et al., “Conditional gene targeting of connexin43: exploring the consequences of gap junction remodeling in the heart,” Cell Commun. Adhes. 8(4-6):345-348 (2001); Vaidya et al., “Null mutation of connexin43 causes slow propagation of ventricular activation in the late stages of mouse embryonic development,” Circ. Res. 88(11):1196-1202 (2001)). Each channel is composed of two identical hexameric structures, “connexons”, that dock across an extracellular space. The result is a permeable pore that is dynamically regulated. The individual subunit of the connexon is the molecule connexin. This molecule resides in the membrane, with its N-terminal (“NT”), cytoplasmic loop (“CL”) and C-terminal (“CT”) domains in the cytoplasmic space. In addition, there are four transmembrane domains and two extracellular domains that are involved in the docking to the opposing connexon. There are at least 20 different connexin isotypes in the mouse genome and 21 in the human genome (Willecke et al., “Structural and functional diversity of connexin genes in the mouse and human genome,” Biol. Chem. 383(5):725-737 (2002)). The most abundant connexin isotype in the heart, brain and other tissues is the 43 KDa protein, Cx43.

Gap junctions allow the passage of ions and small molecules between cells and are regulated by a variety of chemical interactions between the connexin molecule and the microenvironment. As such, gap junctions act as active filters to control the passage of intercellular messages to modulate function.

Previous work has suggested that regulation of Cx43 channels results from the association of the CT domain, acting as a gating particle, and a separate region of the connexin molecule acting as a receptor for the gating particle (Duffy et al., “pH-Dependent Intramolecular Binding and Structure Involving Cx43 Cytoplasmic Domains,” J. Biol. Chem. 277(39):36706-36714 (2002); Moreno et al., “Role of the Carboxyl Terminal of Connexin43 in Transjunctional Fast Voltage Gating,” Circ. Res. 90(4):450-457 (2002), 92(1):e30 (2003) (erratum)). Additional studies have shown that this intra-molecular interaction can be modulated by other inter-molecular interactions in the microenvironment of the gap junction plaque (Morley et al., “Intramolecular Interactions Mediate pH Regulation of Connexin43 Channels,” Biophys. J. 70(3):1294-1302 (1996)). Thus, the emerging picture of a Cx43 gap junction plaque is that of a macromolecular complex where proteins act in concert to modulate intercellular communication. At the center of these interactions is the CT domain, which acts as a substrate for a number of kinases.

Gap junction pharmacology is a nascent field. It has recently been shown that hexa-peptides such as AAP10 (a sequence initially derived from bovine atria), as well as a stable analogue thereof—ZP123, also denoted rotigaptide—and a novel peptide, GAP134, modify gap junctional communication, and show potential as antiarrhythmic agents (Müller et al, Actions of the antiarrhythmic peptide AAP10 on intercellular coupling, Naunyn Schmiedebergs Arch. Pharmacol. 1997 356(1):76-82; Kjolbye et al., Pharmacological modulation of gap junction function with the novel compound rotigaptide: a promising new principle for prevention of arrhythmias. Basic Clin. Pharmacol. Toxicol., 2007 101:215-30; Rossman et al., Effects of the gap junction modifier, GAP-134, on conduction and atrial fibrillation/flutter inducibility in dogs. AHA Scientific Sessions Abstract 1837, 2007 ; Axelsen et al., Increasing gap junctional coupling: a tool for dissecting the role of gap junctions. J. Membr. Biol. 2007;1:23-35).

This accumulated evidence supports the notion of gap junction modification as a relevant pharmacological target. However, further development of these molecules is limited by the fact that their precise molecular target remains undefined, clearly reducing their potential to work as platforms for target-specific drug design.

The present invention was conceived following previous work in which peptide and peptidomimetic compounds were designed on the basis of knowledge of the molecular mechanisms of connexin43 regulation leading to identification of a 34-amino acid peptide, dubbed RXP-E, that is able to bind the carboxyl terminal domain of Cx43 (Cx43CT), to prevent cardiac gap junction closure and also to prevent action potential propagation block. These previous studies allowed for a proof-of-concept regarding the applicability of peptide-based pharmacology to modulation of Cx43 function, though the structure of the core active elements needed for pharmacological development remained undefined. The present inventors therefore used a combination of molecular modelling, surface plasmon resonance and patch clamp strategies to define, for the first time, a unique ensemble of pharmacophores that bind to the carboxyl terminal domain of Cx43 and prevent the closure of Cx43 channels, in particular compounds which are cyclic peptides or cyclic peptidomimetic compounds.

### SUMMARY OF THE INVENTION

Accordingly, in a first aspect, the present invention provides a compound which is a cyclic peptide or a cyclic peptidomimetic compound represented by Formula I

cyclo[Arg¹-Arg²-Pro³-Pro⁴-Tyr⁵-(Arg⁶)ₘ-X⁷] (I)

wherein

m is 0 or 1;

X⁷ is selected from Asn and a Glx group represented by the formula:

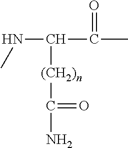

wherein n is 0, 2 or 6

and

X⁷ is linked to Arg¹ via a peptide bond to cyclise the peptide or peptidomimetic compound;

or a compound which differs from Formula I by one, two or three of the following patterns of substitution whereby, if different from Formula I:

the residue at position Arg¹, Arg² and/or Arg⁶ is independently replaced by Lys, His or a lysine mimetic group;

the residue at position Pro³ and/or Pro⁴ is independently replaced by a proline mimetic selected from azetidine, hydroxyproline, morpholino-3-carboxylic acid, and N-substituted amino acids, such as sarcosine, N-cyclohexylglycine, N-phenylglycine, 1-amino-cyclopentane carboxylic acid (Ac5c) or 1-amino-cyclohexane carboxylic acid (Ac6c); and/or

the residue at position Tyr⁵ is replaced by Trp, naphthylalanine , Phe, Met, Val, Ile or Leu;

or a retro analogue or a pharmaceutically acceptable salt thereof.

Preferred combinations of amino acid residue replacements are set out in the following and in the accompanying claims.

In a further aspect, the present invention provides a pharmaceutical composition comprising one or more compounds of the invention together with a pharmaceutical carrier. In a preferred embodiment, the composition is suited for oral administration. The pharmaceutical carrier in a composition of the invention is preferably sterile, pyrogen-free and virus-free.

In a further aspect, the present invention provides a method for modulating gap junctional communication in a population of cells, comprising contacting the population of cells with an amount of a compound of the invention effective to modulate gap junctional communication between the cells. The method may be carried out in vitro, ex vivo or in vivo.

In another aspect, the present invention provides a method of treating a patient having, or at risk of developing, a pathological condition involving impaired gap junctional communication, comprising administering to the patient a therapeutically effective amount of a compound of the invention. The patient will typically be a human, although treatment of non-human animals may also be envisaged.

Examples of pathological conditions involving impaired gap junctional communication include cardiovascular disease, inflammation of airway epithelium, disorders of alveolar tissue, impaired hearing, endothelial lesions, diabetic retinopathy, diabetic neuropathy, ischemia of the central nervous system, ischemia of the spinal cord, dental tissue disorders, osteroporosis, kidney disease, failure of bone marrow transplantation, wounds, erectile dysfunction, urinary bladder incontinence, neuropathic pain, subchronic and chronic inflammation, cancer, transplantation failure, dermal disorders such as psoriasis and conditions caused by an excess of reactive oxygen species and/or free radicals and/or nitric oxide.

A further aspect of the present invention relates to a compound of the invention for use in therapy (i.e. for use in a method of medical treatment). The therapy in question will generally relate to treatment (typically in a human patient) of a pathological condition involving impaired gap junctional communication, e.g. a pathological condition among those as set out above.

A still further aspect of the present invention relates to the use of a compound of the invention in the manufacture of a medicament for the treatment (typically in a human patient) of a pathological condition involving impaired gap junctional communication, e.g. a pathological condition among those as set out above.

Embodiments of the present invention will now be described in more detail, by way of example but not limitation, with reference to the following figures.

### BRIEF DESCRIPTION OF THE FIGURES

FIG. 1. SPR results for compounds (peptides) ZP2769 and ZP2771. Recombinant rat Cx43CT was covalently bound to a carboxylmethyl dextran matrix and used as ligand. Peptides were presented to the bound ligand, and the amplitude of the response was subtracted from that obtained from a control chamber with no ligand. If no change in angle of resonance was observed, this was taken as an indication of absence of binding. The amplitude of the change in angle of resonance (expressed as “response units”) is a function of the molecular mass of the analyte, and for comparison purposes response units were therefore normalized taking into account the molecular weight of the tested compound. Change in angle of incidence of resonance (in “resonance units”) was plotted against time after onset of cyclic peptide exposure. The various curves correspond to different applied concentrations of the peptide in question, as follows:

For ZP2769 (in order, from lowermost to uppermost curve): 63 μM, 125 μM, 250 μM, 500 μM, 1 mM;

For ZP2771 (in order, from lowermost to uppermost curve); 8 μM, 16 μM, 30 μM, 63 μM, 125 μM, 250 μM, 500 μM, 1 mM;

Peptides ZP2769 and ZP2771 both show significant binding to the Cx43CT, with ZP2771 apparently being the more effective.

FIG. 2. Time course of octanol-induced uncoupling in human Cx43-expressing N2A cells. Cell pairs were recorded with patch pipettes that contained either ZP2778 (0.1 mM; upper curve/trace), or only the internal pipette filling solution (control; lower curve/trace). Experiments were conducted using the dual-cell patch clamp technique, and 1.5 mM octanol perfusion was started at Time=0. Inclusion of ZP2778 in the patch pipette significantly (p<0.05) prevented the decrease of Gj compared to that of control.

FIG. 3. Time course of octanol-induced uncoupling in human Cx43-expressing N2A cells. Cell pairs were recorded with patch pipettes that contained either ZP2769 (0.1 mM; upper curve/trace), or only the internal pipette filling solution (control; lower curve/trace). Experiments were conducted using the dual-cell patch clamp technique, and 1.5 mM octanol perfusion was started at Time=0. Inclusion of ZP2769 in the patch pipette resulted in a marked reduction in the decrease of Gj compared to that of control.

FIG. 4. Time course of octanol-induced uncoupling in human Cx43-expressing N2A cells Cell pairs were recorded with patch pipettes that contained either ZP2782 (0.1 mM; upper curve/trace), or only the internal pipette filling solution (control; lower curve/trace). Experiments were conducted using the dual-cell patch clamp technique, and 1.5 mM octanol perfusion was started at Time=0. Inclusion of ZP2782 in the patch pipette resulted in a reduction in the decrease of Gj compared to that of control.

FIG. 5. Time course of octanol-induced uncoupling in human Cx43-expressing N2A cells. Cell pairs were recorded with patch pipettes that contained either ZP2771 (0.1 mM; upper curve/trace), or only the internal pipette filling solution (control; lower curve/trace). Experiments were conducted using the dual-cell patch clamp technique, and 1.5 mM octanol perfusion was started at Time=0. Inclusion of ZP2771 in the patch pipette significantly (p<0.05) prevented the decrease of Gj compared to that for the control, and Gj remained at approximately 0.65 at the end of the experiment compared to a value of Gj=0 for the control after 10 minutes of octanol perfusion.

FIG. 6. Upper graph: Time course of acidification-induced uncoupling in human Cx43-expressing N2A cells. Cell pairs were recorded with patch pipettes that contained either ZP2771 (0.1 mM; upper curve), or only the internal pipette filling solution buffered to pH 6.2 (control; lower curve). Experiments were conducted using the dual-cell patch clamp technique. Inclusion of ZP2771 in the patch pipette significantly (p=0.002) prevented the decrease of Gj compared to that for the control, and Gj remained at approximately 0.49 fifteen minutes (900 sec) after patch break, compared to a value of approximately 0.17 for the control.

Lower graph: Time course of acidification-induced uncoupling in neonatal rat ventricular cardiac myocytes. Cell pairs were recorded with patch pipettes that contained (i) ZP2771 (0.1 mM, buffered to pH 6.2; uppermost curve; filled square symbols), (ii) linear peptide RRPPYR (0.1 mM buffered to pH 6.2; middle curve; filled triangle symbols), or (iii) only the internal pipette filling solution buffered to pH 6.2 (lowermost curve; filled circle symbols). Experiments were conducted using the dual-cell patch clamp technique. Inclusion of ZP2771 in the patch pipette significantly (p=0.006) prevented the decrease of Gj compared to that for the linear peptide RRPPYR and for the control, and Gj remained at approximately 0.47 fifteen minutes after patch break, compared to values of approximately 0.17 and 0.08 for the linear peptide and for the control, respectively, indicating that the cyclic structure of ZP2771, rather than just the net balance of charge, is important for the functional effect of ZP2771 with respect to prevention of low-pH-induced uncoupling.

### DEFINITIONS

Amino acid residues in compounds of the invention may be D- or L-amino acids. In certain embodiments the use of D-amino acids is preferred to enhance stability. Amino acid residues in compounds of the invention are preferably a-amino acids, although in certain embodiments one or more amino acid residues may be β-amino acids (such as azetidine-3-carboxylic acid) or γ-amino acids.

Throughout the description and claims the conventional three-letter or one-letter abbreviations for naturally occurring amino acids are used, as well as generally accepted three letter abbreviations for other α-amino acids, such as sarcosine (Sar), α-amino-iso-butyric acid (Aib), naphthylalanine (Nal) [including 1-naphthylalanine (1Nal) and 2-naphthylalanine (2Nal)], phenylglycine (Phg), 2,4-diaminobutanoic acid (Dab), 2,3-diaminopropanoic acid (Dap), diaminoacetic acid (Daa) and hydroxyproline (Hyp). Where nothing else is specified, Hyp represents 4-hydroxyproline or 3-hydroxyproline. The natural or essential amino acids are the amino acid constituents of proteins. Aromatic amino acids include Phe, Tyr, Trp, 1 Nal, 2Nal and His. Where the L or D form of a particular amino acid residue is not specified, it is to be understood that the residue in question can be in either the L or the D form.

The term “retro analogue” as used in the context of the present invention refers to a peptide whose amino acid sequence is the reverse of that of the compound (a peptide or peptidomimetic compound of the invention) in question.

The term “lysine mimetic” (Lm or LM) as used herein refers to one of the following compounds:

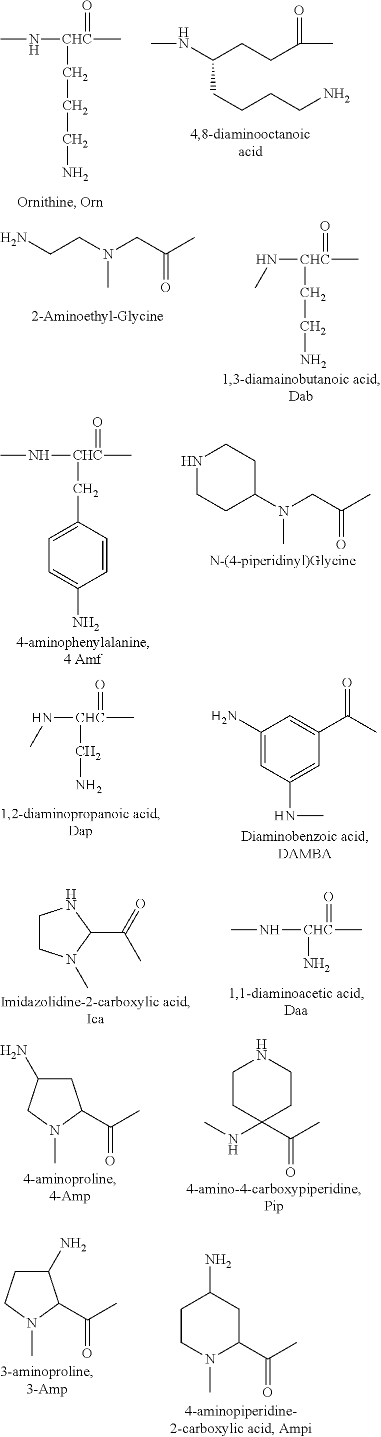

Examples of lysine mimetics having aliphatic cyclic amine groups and aryl amines include Damba, 4-Amp, Ampi, Ica, Pip, and 4AmF, all of which are included above.

3-Aminoproline (3-Amp) and 4-aminoproline (4-Amp) both occur in four isomeric forms (depicted below) as follows:

3-Amp: 2S,3S; 2S,3R; 2R,3S; and 2R,3R;

4-Amp: 2S,4S; 2S,4R; 2R,4S; and 2R,4R.

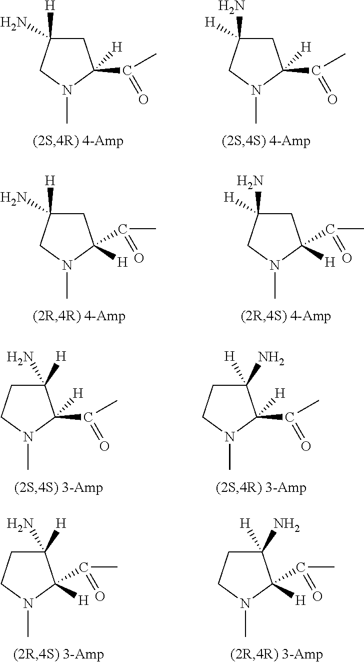

The term “proline mimetic” as used herein refers to one of the following compounds:

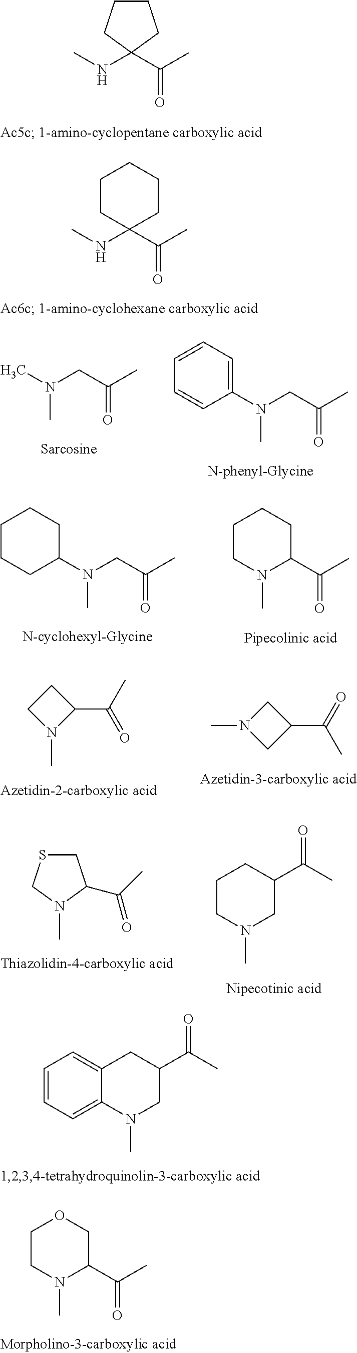

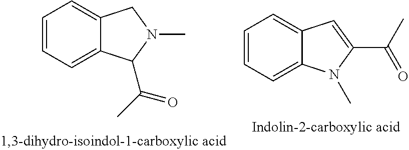

The terms “intercellular communication modulator”, “gap junction facilitator”, “compound that facilitates gap junction communication” and “gap junction opener”, and variants thereof, all refer to a compound that facilitates, maintains or normalizes (e.g. either by inhibiting of enhancing) GJIC, irrespective of the particular mechanism behind this action. More specifically, the term “gap junction opener” may refer to a substance which normalizes (i.e., in general, increases) the exchange of molecules that are able to pass through gap junctions between extracellular and intracellular spaces and/or which can normalize/increase GJIC.

The term “agonist” as used herein refers to a compound (here, cyclic peptide or peptidomimetic) that can interact with a tissue, cell or cell fraction which targeted by, for example—but not limited to—, AAP, AAP10, HP5 peptide or a functional analogue thereof (see, e.g., ??005??? to cause substantially, or at least substantially the same physiological responses in the tissue, cell or cell fractions as that evoked by the AAP, AAP10, HP5 peptide, or functional analogue thereof. By way of example, the physiological response may be one or more of: contraction, relaxation, secretion and enzyme activation. Preferably, the compound binds to the tissue, cell or cell fraction. By way of example, the compound may bind to a receptor on the tissue, cell or cell fraction which binds to AAP, AAP10, HP5 or a functional analog thereof.

The term “antagonist” as used herein refers to a compound (here, cyclic peptide or peptidomimetic) which inhibits or antagonizes one or more physiological responses that are observed in a tissue, cell or cell fraction after contacting the tissue, cell or cell fraction with a compound that targets the tissue, cell or cell fraction in question, such as, for example—but not limited to—, AAP, AAP10, HP5 peptide or a functional analogue thereof. By way of example, the physiological response may be one or more of: contraction, relaxation, secretion and enzyme activation. Preferably, the compound binds to the tissue, cell or cell fraction. By way of example, the compound may bind to a receptor on the tissue, cell or cell fraction which binds to AAP, AAP10, HP5 or a functional analog thereof, and/or may inhibit binding of one or more of AAP, AAP10, HP5 or a functional analog thereof to the receptor.

### DETAILED DESCRIPTION OF THE INVENTION

Before describing aspects of the present invention in greater detail, it should be made clear that the present invention is not to be construed as being limited in any way by the particular methodology, protocols or reagents as described herein.

As discussed above, one aspect of the the present invention relates to peptides that modulate gap junction intercellular communication (GJIC), represented by Formula I as described above. In an embodiment of compounds of the invention, the compound of Formula I is a compound wherein one, two or all three of the amino acid residues Arg¹, Arg² and Arg⁶ independently is replaced with a Lys or His residue, or with a lysine mimetic group, and/or wherein the Tyr⁶ residue is replaced with a Trp, Nal, Phe, Met, Val, Ile or Leu residue.

In a further embodiment, the compound of the invention is a compound wherein the Arg¹ residue is replaced with a Lys or His residue, or with a lysine mimetic group.

In a still further embodiment, the compound of the invention is a compound wherein the Arg² residue is replaced with a Lys or His residue, or with a lysine mimetic group.

In another embodiment, the compound of the invention is a compound wherein the Arg⁶ residue is replaced with a Lys or His residue, or with a lysine mimetic group.

In yet another embodiment, the compound of the invention is a compound wherein the Tyr⁶ residue is replaced with a Trp, Nal, Phe, Met, Val, Ile or Leu residue.

A particular embodiment of the invention relates to a compound of the invention selected from the group consisting of Cyclo(RRPPYQ), Cyclo(RRPPYRQ), Cyclo(RRPPYN) and Cyclo(RRPPWN), or a retro analogue or a pharmaceutically acceptable salt thereof. Each of the latter recited compounds, individually, constitutes an embodiment of a compound of the present invention.

As already indicated above, retro analogues of compounds of Formula I the invention are also within the scope of the present invention. Such retro analogues may be represented by Formula II:

cyclo[X⁷-(Arg⁶)ₘ-Tyr⁵-Pro⁴-Pro³-Arg²-Arg¹] (II)

wherein Arg¹, Arg², Pro³, Pro⁴, Tyr⁶, Arg⁶, X⁷ and m are as defined herein in connection with Formula I (vide supra). Retro analogues of all and any of the embodiments of compounds of Formula I described above are to be understood to be within the scope of the present invention.

Each amino acid residue in a compound of Formula I or Formula II of the invention may is independently be an L-amino acid residue or a D-amino acid residue.

As already indicated above, pharmaceutically acceptable salts of compounds of Formula I or Formula II as set out above are also within the scope of the present invention.

Properties of the Compounds

Compounds of the present invention may be tested for binding to the carboxyl terminal domain of Cx43 using Surface Plasmon Resonance (“SPR”). SPR is a technique that was used to identify the original RXPE sequence (Shibayama et al., “Identification of a Novel Peptide that Interferes with the Chemical Regulation of Connexin-43,” Circ. Res. 98:1365-72 (2006)). The concentration of the compound relative to the amplitude of the response in arbitrary units is measured.

Preferred compounds of the present invention function as effective modulators of gap junctional communication (e.g., as agonists or antagonists) and may therefore be useful, inter alia, as antiarrhythmic drugs.

Medical Uses

As already indicated above, the present invention relates to compounds that are capable of modulating gap junction intercellular communication (GJIC). This may include one or more of the following functions: the ability to decrease cellular uncoupling; to normalize dispersion of action potential duration; to normalize conduction velocity; to control the cellular quantity of gap junctions normalizing (up-regulating or down-regulating, as appropriate) the expression of connexins; to normalize degradation of gap junctions (inhibit or enhance); to normalize cellular trafficking of connexins to the plasma membrane (increase or decrease); to facilitate assembly of connexins into functional gap junctions; to normalize opening of existing gap junctions, e.g., inducing or enhancing opening when they have been closed or gated by inhibitors (such as by mediating or enhancing hyperphosphorylation of the cytoplasmic carboxy terminal domain of one or more connexins (e.g., such as Cx43)), or closing these when they are aberrantly opened (e.g., as in Charcot-Marie-Tooth disease); and the like.

Particular assays useful for identifying and optionally quantifying the activity of the compounds are described below. The assays are non-limiting and serve merely to illustrate a variety of assays in which the present compounds may be tested for their gap junction modulating abilities. It is to be understood that the assays are not mutually exclusive, i.e. a compound may show activity in one particular assay, but show a different, or no, activity in another particular assay.

Arrhythmia

In one preferred aspect, the invention provides compounds having antiarrhythmic properties that may be useful for the treatment of arrhythmias and thrombotic complications arising during cardiovascular disorders, such as acute ischemic heart disease (e.g., stable angina pectoris, unstable angina pectoris, acute myocardial infarction), congestive heart failure (e.g., systolic, diastolic, high-output, low-output, right- or left-sided heart failure), congenital heart disease, corpulmonale(????), cardiomyopathies, myocarditis, hypertensive heart disease, during coronary revascularization(???), and the like.

In specific embodiments, compounds of the invention may be used to treat and/or prevent bradyarrhythmias (e.g., due to disease in sinus node, AV node, bundle of His, right or left bundle branch), and tachyarrhythmias associated with reentry (e.g., atrial premature complexes, AV junctional complexes, ventricular premature complexes, atrial fibrillation, atrial flutter, paroxymal supraventricular tachycardia, sinus node reentrant tachycardia, AV nodal reentrant tachycardia, and non-sustained ventricular tachycardia), either alone or in combination with other antiarrhythmic compounds, such as class I agents (e.g., lidocaine), class II agents (e.g., metoprolol or propranolol), class III agents (e.g., amiodarone or sotalol) or class IV agents (e.g., verapamil).

Additionally or alternatively, compounds of the invention may be used to treat one or more of: a reentry arrhythmia; ventricular reentry (e.g., such as arises during acute myocardial infarction, chronic myocardial infarction, stable angina pectoris and unstable angina pectoris); infectious or autonomic cardiomyopathy; atrial fibrillation; repolarization alternans; monomorphic ventricular tachycardia; T-wave alternans; bradyarrhythmias; and generally, reduced contractility of cardiac tissue, thrombosis and the like.

The antiarrhythmic properties of compounds of the present invention may be tested in an assay measuring the increase in time to an AV block in a mouse after infusion of CaCl₂, in what is referred to herein as a “standard calcium-induced arrhythmia assay”.

Compounds of the present invention may additionally lead to decreases in the incidence of reentry arrhythmias or in the size of an infarct zone observed in what is referred to herein as a “standard ventricular reentry assay”.

Diabetes

There is an understanding that GJIC is important in treatment of diabetes, for example in the treatment of diabetes type 1 or type 2, obesity, eating disorders and insulin resistance syndrome. Compounds of the present invention may improve blood glucose tolerance in diabetic mammals as determined by assays known in the art.

In specific embodiments, compounds of the present invention may be used to stimulate insulin release, lower blood glucose level, reduce gastric motility, delay gastric emptying, inhibit food uptake, e.g. by suppression of appetite, or to lower the plasma lipid level in a vertebrate or a mammal. The compounds may also be used generally in the treatment of diabetes mellitus associated with a risk of hyperglycemia, i.e. where insulin sensitivity is decreased with stress, myocardia infection(???), stroke and infections, or in cases of insulin resistance during pregnancy. The compounds may also be used in the treatment of other types of diabetes, such as cases where diabetes may be secondary to other endocrine diseases, such as acromegaly, Cushing's syndrome, pheochromocytoma, glucagonoma, somatostatinoma, primary aldosteronism, or secondary to administration of certain hormones causing hyperglycemia, or secondary to administration of certain drugs (antihypertensive drugs, thiazide diuretics, preparations containing estrogen, psychoactive drugs, sympathomimetic agents. Furthermore, the compounds may be used generally in the treatment of diseases and conditions associated with a risk of hypoglycemia, i.e. where endogenous glucose production is decreased, such as subsequent to alcohol ingestion, or in cases where the sensitivity to insulin is increased in patients with hypopituitarism or primary adrenocortical insufficiency, or where insulin clearance is decreased (as with progressive renal insufficiency).

Osteoporosis

compounds of the invention may be useful in preventing and/or treating osteoporosis or other pathologies affecting bone formation, growth or maintenance. Compounds which are able to normalize the attenuated GJIC between human osteoblasts during hypoxia are particularly suitable for the treatment of bone diseases in which bone formation is impaired relative to bone resorption. Suitable compounds for use in such methods can be selected in assays for increased alkaline phosphatase (ALP) activity in osteoblasts, which provides a means of monitoring cell viability and growth as a consequence of proper maintenance of GJIC. In one aspect, human osteoblasts are stimulated with different concentrations of peptides from 1×10⁻¹³ to 1×10⁻⁶ mol/l and compared to untreated controls. Under normal culture conditions, compounds preferably increase ALP activity. More preferably, compounds of the invention stimulate ALP activity during hypoxic conditions at concentrations ranging from 10⁻¹¹ to 10⁻⁸ mol/l. This can thus be used to optimize compound selection and design for the treatment and/or prevention of bone diseases associated with poor vascularization, hypoxia and ischemia in bone tissue.

Compounds of the present invention may also be used for the prevention and/or treatment of joint diseases that involve impaired cell-to-cell coupling. This may include their use for the prevention and/or treatment of joint diseases that involve metabolic stress, for example forms of arthritis associated with decreased vascularization, or healing of fractured cartilage tissue.

The function of compounds of the invention in modulating GJIC in bone formation may be assessed using an assay in which the compound are tested to determine whether they increase osteoblast activity in a “standard osteoblast activity assay” which measures either calcium wave formation and/or alkaline phosphatase activity of osteoblast cells in the presence of the compounds. Preferably, such compounds increase calcium wave activity, manifested as an increase in the number of cells involved in a wave (as determined by measuring levels of intracellular Ca²⁺ using a calcium sensitive fluorescent dye, such as fura-2, and counting the number of cells which fluoresce). Alkaline phosphatase activity can also be used to provide a measure of osteoblast activity using standard colorimetric assays.

Wounds

Compounds of the invention may be useful in treating wounds and, in particular, in accelerating wound healing. Wound healing involves the interactions of many cell types, and intercellular communication mediated by gap junctions is considered to play an important role in the coordination of cellular metabolism during the growth and development of cells involved in tissue repair and regeneration (K. M. Abdullah, et al. (1999) Endocrine, 10 35-41; M. Saitoh, et al. (1997) Carcinogenesis, 18: 1319-1328; J. A. Goliger, et al. (1995) Mol. Biol. Cell, 6 1491-1501). The compounds may be administered at the site of a wound by topical administration using carriers well known in the art (e.g., ointments, creams, etc.), or may administered systemically, e.g., for treating wounds in internal tissues, such as in the treatment of chronic gastric ulcer lesions.

Additional functions in which endothelial gap-junctional intercellular communication has been implicated are the migratory behavior of endothelial cells after injury, angiogenesis, endothelial growth and senescence, and the coordination of vasomotor responses (G. J. Christ, et al. (2000) Braz. J Med Biol. Res., 33: 423-429). Thus, compounds of the invention may be useful in enhancing conducted vascular responses and/or improving blood supply under conditions involving increased metabolic demand (e.g., physical exercise, tachycardia), and during ischemia.

Gap junctions are also believed to provide a molecular link for coordinated long-range signaling among individual members of the glial compartment. Likewise, astrocytes are ideally suited for metabolic support of neurons since they are functionally polarized with one extremity touching the vascular bed, and the other pole approximates neuronal parenchyma (R. Dermietzel (1998) Brain Res. Brain Res. Rev., 26: 176-183). Thus, the compounds of the present invention may be administered to a patient in need thereof to prevent ischemic damage in the brain by increasing the metabolic support between glia cells and neurons. Such patients may include patients with organic psychoses, who may present with symptoms such as depression, anxiety, learning and memory deficit, phobias, and hallucinations, and patients who have suffered a traumatic brain injury. For such applications, the compounds are preferably selected or formulated so as to be available to the central nervous system (i.e. provided with, or conjugated with, carriers which facilitate transport across the blood-brain barrier).

Compounds according to the invention may also be used to accelerate repair after nerve injury, or during grafting of immature cells (progenitor cells) into brain tissue, such as in patients with neurotrauma, brain ischemia or chronic neurodegenerative disease, such as Parkinson's disease or Huntington's disease (H. Aldskogius, et al. (1998) Prog. Neurobiol, 55: 1-26).

In addition, compounds of the present invention may, due to their effect on intercellular gap junction channels, be used to treat and/or prevent cataract (D. Mackay, et al. (1999) Am J Hum. Genet, 64 1357-1364), to treat and/or prevent vascularization of the cornea in disease states with poor nutrition of the cornea, to increase the healing of corneal lesions (S. G. Spanakis, et al. (1998) Invest Ophthalmol. Vis. Sci., 39: 1320-1328), and/or to prevent hypertension.

Psoriasis

In a further aspect, compounds of the invention may be useful in the treatment of psoriasis. Psoriasis is a complex multifactorial condition resulting in characteristic psoriatic plaques and lesions. It is characterised by disordered epidermal cell proliferation and differentiation. There is a strong association with susceptibility to the epidermal differentiation complex—a locus of up to 50 genes with roles in epidermal differentiation—, suggesting that impaired barrier function, or recovery seem to contribute to the progression of the psoriatic state (Capon et al.,2001; Djalilian et al 2006).

Central to maintenance of healthy skin is a tight and highly ordered cell-to-cell adhesion network including adherens, desmosomes and gap junction intercellular communication channels—the epidermal junctional nexus (Laird, 2006; Prochnow and Dermietzel, 2008). These intercellular junctions play crucial roles in epidermal proliferation, migration and differentiation and are all altered to differing degrees during psoriasis. Expression of connexins (Cx), the constituent proteins of gap junctions, is spatially and temporally regulated within the epidermis with Cx43 in basal and spinous layers normally being replaced by Cx26 in the upper granular layers (Di et al., 2001). In psoriatic plaques, however, Cx26 is greatly increased throughout the epidermis in a pattern similar to that in other hyperproliferative, stratified epithelia. In addition to the spatial redistribution of Cxs found in psoriasis there is also an associated down regulation of E-cadherin in the basal cell and upper spinous layers compared to normal epidermis (Chung et al., 2005). Thus, the hyperproliferation and altered differentiation status of psoriatic keratinocytes is associated with extensive remodelling of both gap junctions and adherens junctions. The carboxyl terminal tails of Cx43, and cadherins, are subject to post translational modification, particularly phosphorylation, and interact with a variety of proteins including ZO-1 and β-catenin that serve as a link between the membrane incorporated protein and the cytoskeletal network (Laird, 2006; Prochnow and Dermietzel, 2008). Compounds of the invention are known to maintain and regenerate Cx43 GJIC under stress conditions and may therefore be used for the treatment of psoriasis, inhibiting the down-regulation of Cx43 and replacement with Cx26.

Cancer

In a further aspect, compounds of the invention may be useful in the treatment of cancer. Carcinogenesis is characterized by the progressive impairment of growth control mechanisms in which growth factors, oncogenes and tumor suppressor genes are involved. A general theme in carcinogenesis and tumorigenesis is down-regulation of the GJIC. Permeability of gap junctions in tumor cells, determined using the dye-transfer assay, is typically lower than GJIC in surrounding tissue. Further, the gating of gap junctions is known to be affected/effected??? by tumor promoters, which decrease GJIC. This means that compounds of the present invention may useful as medicaments for the treatment of cancer, either used alone or in conjunction with traditional anti-cancer therapies.

The suitability of compounds of the invention for treating cancer may, for example, be assessed using an assay in which the decrease of GJIC inhibition mediated by tumor promoters, such as DTT, is determined. Using this “standard tumor promoter assay”, compounds will preferably show decreases in GJIC inhibition which are at least 50%, preferably 70%, more preferably 100% or more of decreases observed for AAP.

Pharmaceutical Compositions

The compounds of the present invention may be formulated as pharmaceutical compositions comprising one or more of compounds of the invention, in combination with a pharmaceutically acceptable carrier and/or diluent. Such compositions are preferably in a form adapted for oral administration. Formulations for oral administration may be prepared in a manner well known to the person skilled in the art, e.g., as generally described in “Remington's Pharmaceutical Sciences”, 17. Ed. Alfonso R. Gennaro (Ed.), Mark Publishing Company, Easton, Pa., U.S.A., 1985 and more recent editions, and in the monographs in the “Drugs and the Pharmaceutical Sciences” series, Marcel Dekker. By way of example, compositions may be in the form of tablets, capsules, granules, pellets, troches, lozenges, and the like. Suitable enteric formulations are described, e.g., in U.S. Pat. No. 5,350,741.

The pharmaceutical carrier or diluent employed may be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax.

If a solid carrier is used for oral administration, the preparation may be tabletted, placed in a hard gelatin capsule in powder or pellet form, or it can be in the form of a troche or lozenge. The amount of solid carrier will vary widely but will usually be from about 25 mg to about 1 g. If a liquid carrier is used, the preparation may be in the form of a syrup or liquid suitable for oral ingestion.

It will be appreciated that the actual preferred amounts of a compound used in a given therapy will vary according to, e.g., the specific compound being utilized, the particular composition formulated, the mode of administration, and the nature and characteristics of the subject to be treated, e.g. the species, sex, weight, age and general state of health the subject. Optimal administration regimens for a given protocol of administration can be readily ascertained by those skilled in the art using conventional dosage determination tests conducted with regard to the foregoing guidelines. Suitable dose ranges may include from about 1 mg/kg to about 100 mg/kg body weight per day.

Compounds of the present invention are suitably administered in a protonated and water-soluble form, e.g., as a pharmaceutically acceptable salt, typically an acid addition salt such as an inorganic acid addition salt, e.g., a hydrochloride, sulfate, or phosphate salt, or as an organic acid addition salt such as an acetate, trifluoroacetate, maleate, fumarate, tartrate or citrate salt. Pharmaceutically acceptable salts of therapeutic compounds of the invention may also include metal salts, particularly alkali metal salts such as a sodium salt or potassium salt; alkaline earth metal salts such as a magnesium or calcium salt; ammonium salts such an ammonium or tetramethylammonium salt; or amino acid addition salts such as a lysine, glycine, or phenylalanine salt.

Compounds of the invention may also be administered topically to treat peripheral vascular diseases, and as such may be formulated as a cream or ointment.

Compounds of the invention may also be formulated in compositions such as sterile solutions or suspensions for non-oral administration, e.g. by parental administration, such as by intravenous, intramuscular, subcutaneous, intranasal, intrarectal, intravaginal or intrapritoneal administration.

Compounds and compositions of the invention may be used to treat conditions or pathologies associated with impaired (abnormal decreases or increases in) gap junctional communication. In accordance with normal clinical practice, the compounds or compositions are administered to a patient in a therapeutically effective amount. As used herein, “a therapeutically effective amount” is one which reduces symptoms of a given condition or pathology, and preferably which normalizes physiological responses in a patient with the condition or pathology. Reduction of symptoms or normalization of physiological responses can be determined using methods routine in the art and may vary with according to the particular condition or pathology.

Examples of conditions which may be treated using compounds or compositions of the invention include, but are not limited to, cardiovascular disease, inflammation of airway epithelium, disorders of alveolar tissue, bladder incontinence, impaired hearing due to diseases of the cochlea, endothelial lesions, diabetic retinopathy and diabetic neuropathy, ischemia of the central nervous system and spinal cord, dental tissue disorders including periodontal disease, kidney diseases, failures of bone marrow transplantation, wounds, erectile dysfunction, urinary bladder incontinence, neuropathic pain, subchronic and chronic inflammation, cancer and failures of bone marrow and stem cell transplantation, conditions which arise during transplantation of cells and tissues or during medical procedures such as surgery; as well as conditions caused by an excess of reactive oxygen species and/or free radicals and/or nitric oxide.

Methods

Peptide Synthesis of Cyclic Compounds

A preferred general peptide synthesis procedure is described below. However, a more detailed description of solid-phase peptide synthesis methodology may be found in, for example, WO 01/62775:

Peptides are synthesized batchwise in a polyethylene vessel equipped with a polypropylene filter for filtration, using 9-fluorenylmethyloxycarbonyl (Fmoc) as N-α-amino protecting group and suitable common protection groups for side-chain functionalities. All couplings are continued for at least 2 hours. All cyclizations are performed as described in WO 01/62775. Acylations are checked by the ninhydrin test, performed at 80° C. as described in WO 01/62775???. After completed synthesis the peptide-resin is washed with DMF (3×15 ml, 1 min each), DCM (3×15 ml, 1 min each), diethyl ether (3×15 ml, 1 min each) and dried in vacuo. The peptide is then cleaved from the resin as described in WO 01/62775?? and freeze-dried. After purification using preparative HPLC as described WO 01/62775???, the peptide product is collected and the identity of the peptide is confirmed by ES-MS.

Surface Plasmon Resonance (SPR)

SPR is a spectroscopic technique that permits determination of binding amplitude and kinetics in real time (Salamon et al., “Surface Plasmon Resonance Spectroscopy as a Tool for Investigating the Biochemical and Biophysical Properties of Membrane Protein Systems. II: Applications to Biological Systems,” Biochim. Biophys. Acta 1331(2):131-152 (1997); Duffy et al., “Functional Demonstration of Connexin-protein Binding Using Surface Plasmon Resonance,” Cell Adhes. Commun. 8(4-6):225-229 (2001); Lang et al., “Surface Plasmon Resonance as a Method to Study the Kinetics and Amplitude of Protein-protein Binding,” in Practical Methods in Cardiovascular Research 936-947 (Stefan Dhein, Friedrich Wilhelm Mohr & Mario Delmar eds., 2005)). Recombinant Cx43CT is covalently bound to a carboxylmethyl dextran matrix (Salemon et al., “Surface Plasmon Resonance Spectroscopy as a Tool for Investigating the Biochemical and Biophysical Properties of Membrane Protein Systems. II: Applications to Biological Systems,” Biochim. Biophys. Acta 1331(2):131-152 (1997)). Specific peptide compounds are presented and, when feasible, dissociation constants (KD) are calculated from the time course of binding and unbinding of the ligand, using a 1:1 (Langmuir) association and dissociation kinetic model (Biacore software package). In both phases (association and dissociation), the first 5-8 seconds of recording are not included in the fit, to avoid artifacts resulting from peptide distribution within the flow cells (Lang et al., “Surface Plasmon Resonance as a Method to Study the Kinetics and Amplitude of Protein-protein Binding,” in Practical Methods in Cardiovascular Research 936-947 (Stefan Dhein, Friedrich Wilhelm Mohr & Mario Delmar eds., 2005). In the present experiments recombinant rat Cx43CT was covalently bound to a carboxylmethyl-dextran matrix and used as ligand.

Electrophysiological Analysis.

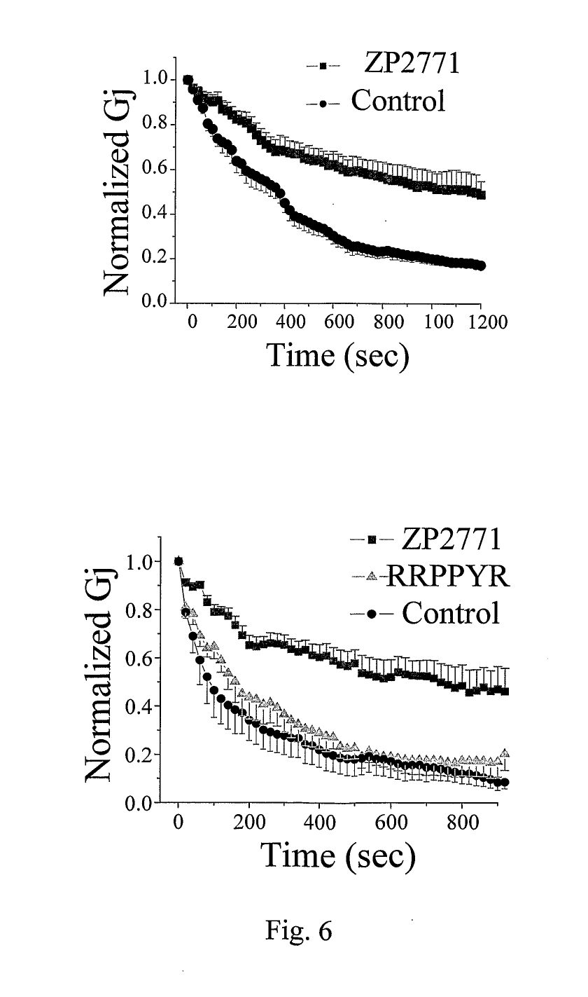

Patch clamp experiments were conducted in N2a cells transiently transfected with Cx43. In all cases, the dual-whole-cell voltage clamp technique was used to record gap junction currents. Specifics for cell culture, transfection and recordings are described in Shibayama et al. (Identification of a novel peptide that interferes with the chemical regulation of connexin 43. Circ Res 2006 98:1365-72). Octanol superfusion was initiated 5 minutes after patch break and continued for 10 minutes. Concentration of octanol was 1.5 mM in all experiments. Peptides were diluted in the internal pipette solution to a final concentration of 100 μM. A total of 20 experiments were carried out where octanol-induced uncoupling was tested in the absence of peptides. These experiments were averaged, and data used as control for comparison with those series where a given peptide was assessed. Patch clamp experiments were also conducted (cf. FIG. 6 herein) using ventricular myocytes from neonatal Sprague-Dawley rats (rats supplied by Charles River, Mass. USA), isolated and cultured according to Rohr et al. (see Circ. Res. 1991; 68: 114-130, and J. Cardiovasc. Electrophysiol. 1995; 6: 551-568).

Quantitative Analysis of Cx43CT-Compound Interactions.

The screening method described in part 1, below (??OK??), allows identification of compounds that do not bind to Cx43CT. A preliminary ranking order may be assigned to those compounds that bind to the target. Part 2, below, describes how more detailed quantitative parameters may be established to define the Cx43CT-compound interaction.

Specific Experiments:

1) Determination of Cx43CT-compound dissociation constant. The binding kinetics of each compound to Cx43CT is assessed by SPR. Various concentrations of ligate are used, and the rate of association and dissociation is fitted with a Langmuir 1:1 kinetic model (Biacore). This model assumes first-order kinetics of binding. Proper fitting will yield an estimation of KD. In some cases, the interaction of the compound with the Cx43CT may depart from a uni-uni model, in which case KD values are not generated.

2) Concentration-dependence of binding using cross-linking reagents. This system allows the establishment of a quantitative parameter for compound-Cx43CT association even if the binding kinetics are not suitable for KD determinations. Cross-linking is tested for a constant concentration of Cx43CT and sequential dilutions of the compound. The ratio of bound versus unbound densities is plotted as a function of compound concentration. The peptide concentration corresponding to half-maximum binding is defined as the apparent EC₅₀.

3) Identification of compound-induced Cx43CT resonance shifts by nuclear magnetic resonance (NMR). Previous studies have identified those amino acids in Cx43CT whose position in space is affected by RXP4 and by RXPE (EVT.REFERENCE????). The resonance map for Cx43CT is repeated with candidate compounds.

### EXPERIMENTAL EXAMPLES

The invention will now be further illustrated with reference to the following examples. It will be appreciated that what follows is by way of example only, and that modifications may be made while still falling within the scope of the invention.

### EXAMPLES

### Example 1

### Peptide Synthesis of Cyclic Compounds

The following compounds of Table 1 were synthesized and representative examples of them tested in the following examples.

TABLE 1
Sequence and MW of synthesized compounds

| | | MW |
|---|---|---|
| | | average |
| ZP # | Sequence | (g/mol) |
| 2769 | Cyclo(RRPPYQ) | 797.92 |
| 2771 | Cyclo(RRPPYRQ) | 954.11 |
| 2778 | Cyclo(RRPPYN) | 783.89 |
| 2782 | Cyclo(RRPPWN) | 806.93 |

### Example 2

### Surface Plasmon Resonance (SPR)

Examples of the cyclic peptide compounds of the present invention were presented to the bound ligand, and the amplitude of the response was subtracted from that obtained from a control chamber with no ligand. No change in angle of resonance was taken as an indication of absence of binding. The amplitude of the change in angle of resonance (expressed as “response units”) is a function of the molecular mass of the analyte and for comparison purposes response units were normalized taking account of the molecular weight of the tested compound. Change in angle of incidence of resonance (in “resonance units”) was plotted against time after onset of cyclic peptide exposure.

TABLE 2
SPR data for cyclic peptides at different concentrations.

| Peptide | Mw | Conc | Resonance | |
|---|---|---|---|---|
| ZP # | g/mol | mM | Units (ru) | (ru * 100)/Mw |
| 2769 | 797.92 | 1.000 | 196.2 | 24.59 |
| | 797.92 | 0.500 | 101.9 | 12.77 |
| | 797.92 | 0.250 | 55.8 | 6.99 |
| | 797.92 | 0.125 | 29.6 | 3.71 |
| | 797.92 | 0.063 | 18.6 | 2.33 |
| 2771 | 953.53 | 1.000 | 1253.0 | 131.41 |
| | 953.53 | 0.500 | 896.0 | 93.97 |
| | 953.53 | 0.250 | 574.0 | 60.20 |
| | 953.53 | 0.125 | 323.6 | 33.94 |
| | 953.53 | 0.063 | 192.4 | 20.18 |
| | 953.53 | 0.031 | 95.8 | 10.05 |
| | 953.53 | 0.016 | 58.1 | 6.09 |
| | 953.53 | 0.008 | 27.6 | 2.89 |

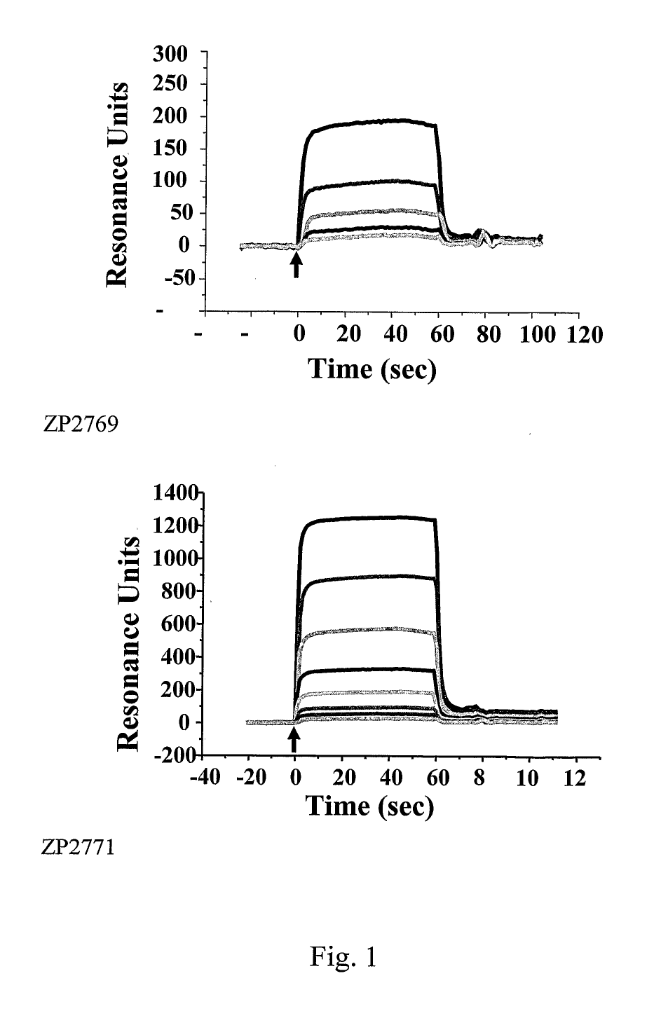

SPR allows for assessment of ligand-analyte binding in real time, and was used for characterization of two of the cyclic peptide compounds of the present invention. Cx43CT was covalently bound to the matrix of a sensor chip. Table 2 shows the change in angle of incidence of resonance was recorded, thus indicating direct binding of cyclic peptide compounds to Cx43CT. In FIG. 1, the change in angle of incidence of resonance (in “resonance units”) is plotted against time after onset of cyclic peptide exposure. Different concentrations were applied, as indicated. It is apparent that both ZP2769 and ZP2771 show significant binding to the Cx43CT, although ZP2769 seems to be less effective.

### Example 3

### Electrophysiological Analysis

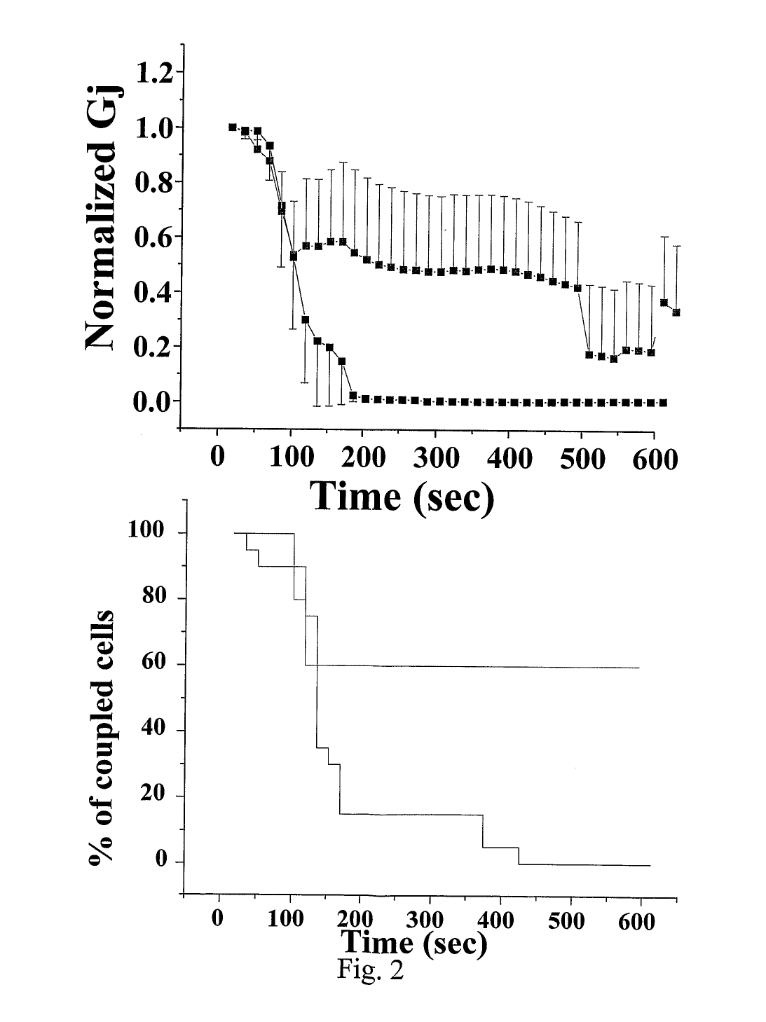

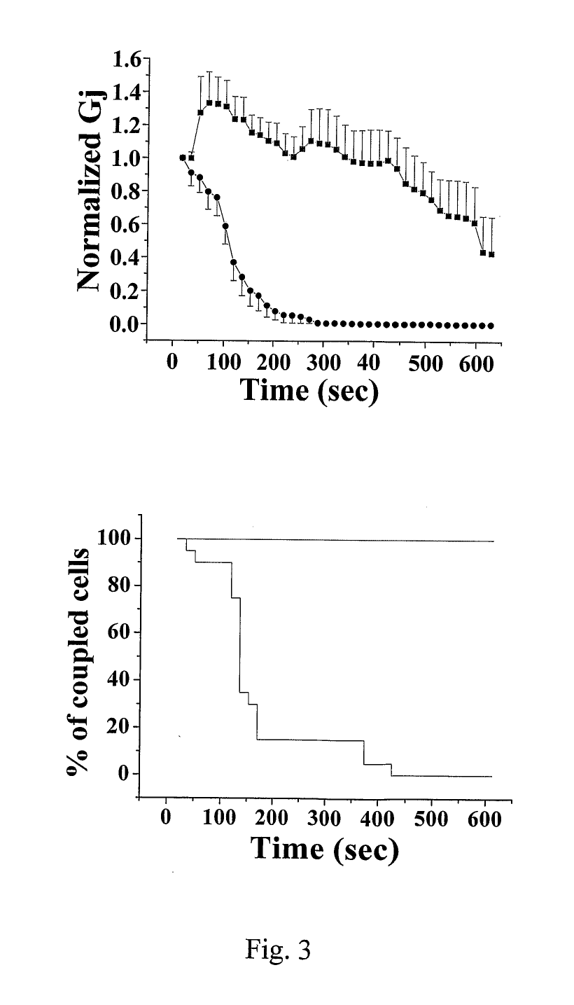

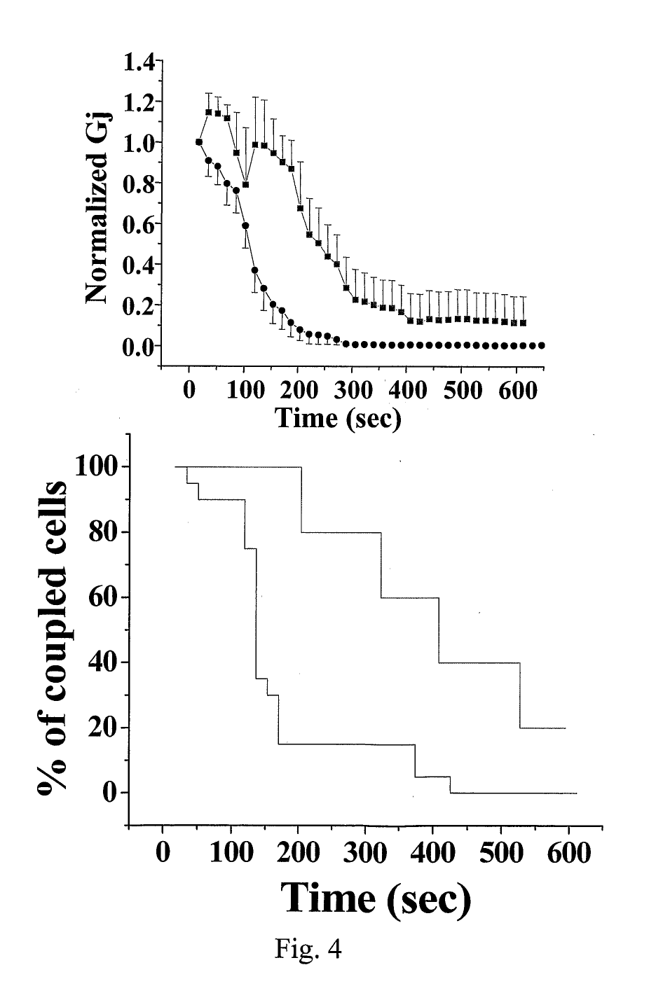

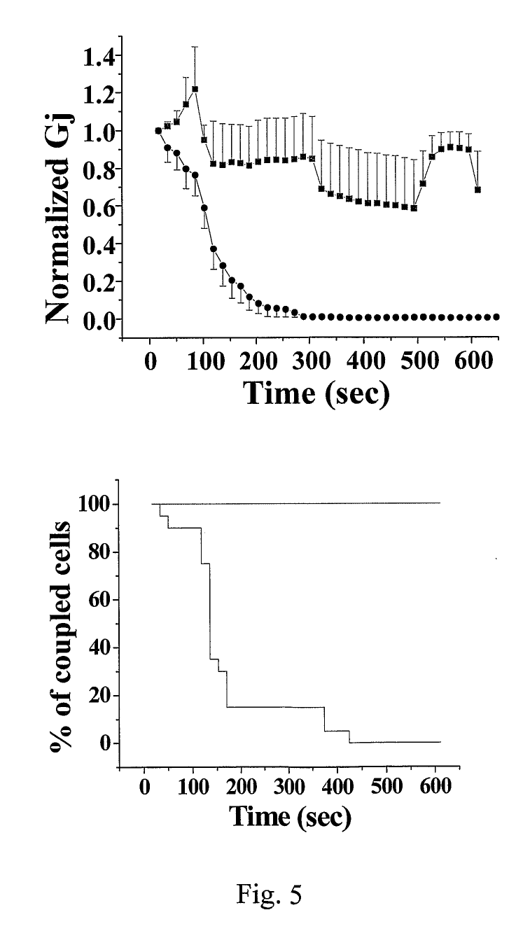

Junctional conductance (Gj) was measured in N2a cell pairs expressing Cx43. Patch pipettes were filled with an internal solution containing the specific peptide under study, and the time course and extent of octanol-induced uncoupling was compared to that observed in the absence of the peptide. FIGS. 2-5 show average measurements of junctional conductance and percent of coupled cells recorded at various times after the onset of octanol superfusion for the cyclized peptides ZP2769, ZP2771, ZP2778, ZP2782 and for the dissolution medium (internal pipette solution) used as control. The data show that, in the presence of the cyclized peptides, the progression of octanol-induced uncoupling was significantly delayed and the minimum value of average Gj recorded at the end of the 10-minute octanol exposure was significantly different from that obtained in the absence of the peptides (p<<0.05). Moreover, for the peptides ZP2769 (FIG. 3) and ZP2771 (FIG. 5), all cells remained coupled ten minutes after the onset of octanol superfusion. For ZP2778 (FIG. 2), 60% of the cells remained coupled, while 20% remained coupled for ZP2782 (FIG. 4).

1. A compound which is a cyclic peptide or a cyclic peptidomimetic compound represented by Formula I
cyclo[Arg¹-Arg²-Pro³-Pro⁴-Tyr⁵-(Arg⁶)ₘ-X⁷] (I)
wherein
m is 0 or 1
X⁷ is selected from Asn or a Glx group represented by the formula: 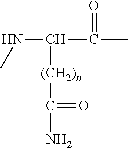
and n is 0, 2 or 6, and
X⁷ is linked to Arg1 via a peptide bond to cyclise the peptide or peptidomimetic compound;
or a compound which differs from Formula I at one, two or three of the following positions, whereby, if different from Formula I:
the residue at position Arg¹, Arg² and/or Arg⁶ is independently replaced by Lys, His or a lysine mimetic group;
the residue at position Pro³ and/or Pro⁴ is independently replaced by a proline mimetic selected from azetidine, hydroxyproline, morpholino-3-carboxylic acid or an N-substituted amino acid such as sarcosine, N-cyclohexylglycine or N-phenylglycine or 1-amino-cyclopentane carboxylic acid (Ac5c) or 1-amino-cyclohexanecarboxylic acid (Ac6c);
the residue at position Tyr⁵ is replaced by Trp, naphthylalanine , Phe, Met, Val, Ile or Leu;
or a retro analogue thereof or a pharmaceutically acceptable salt thereof.

2. The compound according to claim 1, wherein the compound comprises one, two or three changes in which the residue at position Arg¹, Arg² and/or Arg⁶ is independently replaced by Lys, His or a lysine mimetic group and/or the residue at position Tyr⁵ is replaced by Trp, naphthylalanine , Phe, Met, Val, Ile or Leu.

3. The compound according to claim 1, wherein the compound comprises a change at position Arg¹ in which the residue is replaced by Lys, His or a lysine mimetic group.

4. The compound according to claim 1, wherein the compound comprises a change at position Arg² in which the residue is replaced by Lys, His or a lysine mimetic group.

5. The compound according to claim 1, wherein the compound comprises a change at position Arg⁶ in which the residue is replaced by Lys, His or a lysine mimetic group.

6. The compound according to claim 1, wherein the compound comprises a change at position Tyr⁵ in which the residue is replaced by Trp, naphthylalanine , Phe, Met, Val, Ile or Leu.

7. The compound of claim 1, which is Cyclo(RRPPYQ), Cyclo(RRPPYRQ), Cyclo(RRPPYN), Cyclo(RRPPWN), or a retro analogue thereof or a pharmaceutically acceptable salt thereof.

8. A pharmaceutical composition comprising one or more compounds according to claim 1 and a pharmaceutical carrier.

9. The pharmaceutical composition according to claim 8, wherein the composition is orally administrable.

10. A method for modulating gap junctional communication in a population of cells comprising administering an effective amount of a peptide according to claim 1 to a population of cells thereby modulating gap junctional communication between the cells.

11. A compound according to claim 1 for use in method of medical treatment.

12. The compound according to claim 11, wherein the medical treatment is selected from the group consisting of a cardiovascular disease, inflammation of airway epithelium, a disorder of alveolar tissue, impaired hearing, an endothelial lesion, diabetic retinopathy, diabetic neuropathy, ischemia of the central nervous system, ischemia of the spinal cord, a dental tissue disorder, osteroporosis, kidney disease, failure of bone marrow transplantation, wound, erectile dysfunction, urinary bladder incontinence, neuropathic pain, subchronic and chronic inflammation, cancer, transplantation failure; dermal disorders such as psoriasis and conditions caused by an excess of reactive oxygen species and/or free radicals and/or nitric oxide.

13. A method of treating a patient having a pathological condition involving impaired gap junctional communication, said method comprising administering to said patient a compound of claim 1.

14. The method according to claim 13, wherein the patient is a human being.

15. The method according to claim 13, wherein the pathological condition is selected from the group consisting of a cardiovascular disease, inflammation of airway epithelium, a disorder of alveolar tissue, impaired hearing, an endothelial lesion, diabetic retinopathy, diabetic neuropathy, ischemia of the central nervous system, ischemia of the spinal cord, a dental tissue disorder, osteroporosis, kidney disease, failure of bone marrow transplantation, wound, erectile dysfunction, urinary bladder incontinence, neuropathic pain, subchronic and chronic inflammation, cancer, transplantation failure; dermal disorders such as psoriasis and conditions caused by an excess of reactive oxygen species and/or free radicals and/or nitric oxide.
